(19) United States
(12) Patent Application Publication
(10) Pub. No.: US 2020/0390577 A1
(43) Pub. Date: Dec. 17, 2020

(54) METHOD AND SYSTEM FOR DETECTING VOLUNTARY BINARY RESPONSES BY ANALYZING THE PUPIL DIAMETER OF A SUBJECT

(71) Applicant: UNIVERSITÀ DEGLI STUDI DI TORINO, Torino (IT)

(72) Inventors: Silvestro ROATTA, Bastia Mondovì (Cuneo) (IT); Claudio DE'SPERATI, Torino (IT); Luca MESIN, (Avigliana) Torino (IT); Valentina PASIAN, Torino (IT); Francesco PONZIO, Torino (IT)

(21) Appl. No.: 16/763,181

(22) PCT Filed: Nov. 12, 2018
(86) PCT No.: PCT/IB2018/058856

§ 371 Date: May 11, 2020

(30) Foreign Application Priority Data
Nov. 14, 2017 (IT) 102017000129971

(51) Int. Cl. A61F 4/00; G06F 3/01; G09B 21/00

(52) U.S. Cl. CPC A61F 4/00; A61B 3/0091; G09B 21/00; G06F 3/013

(57) ABSTRACT

A method is described for detecting voluntary binary responses of a subject affected by partial or total paralysis of skeletal muscles. The method comprises arrangement of at least one visual target (14) in a position proximal to the subject, and at least one visual target (16) in a position distal to the subject. At least one question is posed to the subject, requiring an answer that is either affirmative or negative. Then, a value is recorded (2) identifying the diameter of the subject's pupil during a first time-interval (R1) and, optionally, during a second time-interval (R2), in which the pupil diameter may reduce in size, due to the shift of focus from the distal target (16) to the proximal target (14), performed by the subject. A computer (24) may, therefore, classify the response to the question as being affirmative or negative, depending on whether, by processing the recorded values, a reduction in pupil diameter is detected in the first interval or in the second interval, respectively.

### FIELD OF THE INVENTION

The present description refers in general to methods and systems for the eye tracking of subjects affected by diseases of the nervous system. More specifically, the description concerns methods and systems for detecting voluntary binary responses of subjects whose partial or total paralysis of skeletal muscles affects their ability to communicate.

### BACKGROUND

In many pathologies of the nervous system (for example, amyotrophic lateral sclerosis, cerebral infarction, and intoxication by botulinum toxin), complete paralysis of the skeletal muscles prevents the subject from communicating with the outside world, despite maintaining a state of vigilance and consciousness internally (patients considered to be in locked-in status and complete locked-in status, LIS and CLIS, respectively).

Methodologies and technologies based on decoding the brain activity (commonly known as Brain-Computer-Interfaces, BCI) have been proposed with the aim of identifying the subject's intentions.

Among the non-invasive BCI systems, those based on the analysis of electroencephalogram (EEG) signals are particularly promising, being quite user-friendly. The results obtained so far are, however, limited and with still sub-optimal success rates.

One of the greatest difficulties of these systems is related to the high variability and very noisy nature of the EEG, which makes analysis and interpretation of the results extremely complex and weak.

Another difficulty is due to the complexity of the mental task required of the patient, which often requires long ad-hoc training (for example, motor imagination tasks).

Other BCI systems, for example, those based on magneto-encephalography (MEG) or functional magnetic resonance imaging (fMRI), are not suitable for current use, e.g., as a support for domestic aid, due to the cost and complexity of use.

There are also known methods (for example from EP 1 164 919 B1) for detecting the cognitive activity of a subject based on the detection of changes in the pupil diameter.

Pupil size depends on the extension of the iris, which is governed by smooth (non-skeletal) muscles, under the control of the sympathetic and parasympathetic vegetative nervous system.

Changes in the pupillary diameter detected—to date—in order to open a communication channel with the patient, constitute operative methods that require important cognitive efforts by the subject. For example, in a study published in 2013, subjects were asked to perform a challenging mental calculation, which is a stressful condition that produces pupillary dilation (Stoll et al., 2013).

In another study published in 2014, healthy volunteers were asked to shift their visual-spatial attention, while maintaining ocular fixation in a central position (Binda et al., 2014; Mathot et al., 2016).

In all the aforesaid studies, the difficulty of the task represented a non-marginal element of the method of evaluation and interpretation of the ocular response; moreover, the subjects were required to train and become familiar with the cognitive task.

### SUMMARY OF THE INVENTION

The present description aims to provide new methods and systems to identify and detect the intentions of a subject whose partial or total paralysis of the skeletal muscles prevents communication.

According to the present description, the above object is achieved thanks to the subject-matter specifically recalled in the following claims, which are understood as forming an integral part of this disclosure.

One embodiment of the present disclosure provides a method for detecting voluntary binary responses of a subject, e.g., a subject suffering from partial or total paralysis of the skeletal muscle, comprising the following steps;
arranging at least one visual target proximal to the subject and at least one visual target distal to the subject,
submitting the subject to at least one question that requires a binary response, e.g., affirmative answers (“YES”) or negative ones (“NO”).

For example, in various embodiments, the subject may be instructed to respond by looking at the proximal visual target (e.g., to provide an affirmative response) or at the distal target (e.g., to provide a negative response) during a given time-interval. Preferably, the subject is instructed to look at the distal target while the question is submitted to him.

Therefore, in various embodiments, the method may include the following steps:
recording an identifying value of the pupil diameter of the subject during a time-interval, in which the pupil diameter is subject to reduction because of the shift in attention, due to focusing, from the distal to the proximal visual target,
classifying the answer to the question as either affirmative or negative by processing the recorded value, by detecting if the pupil diameter presents a reduction in the aforesaid time-interval.

In different embodiments, a second time-interval of verification may be provided (in addition to the previous time-interval, mentioned above). In this case, the subject may be instructed to respond by looking at the proximal target during a first interval and the distal target during a (consecutive) second interval (or example, to provide an affirmative response), or vice versa, by looking at the distal target during the first interval and the proximal target during the second (for example, to provide a negative response).

In this case, the method can therefore include the following steps:
recording the diameter of the pupil during the (consecutive) second time-interval as well, and
classifying the answer to the question as either affirmative or negative by processing the recorded value, detecting if the pupil diameter presents a reduction in either the first or in the second time-interval.

Preferably, the method can verify if the pupil diameter presents a reduction only in the first time-interval or only in the second time-interval.

For example, in various embodiments, two identifying values of the pupillary diameter are determined during the first time-interval and during the second time-interval, respectively. In the case in which only the first time-interval is used, the second value of the pupil diameter can be detected while the question is posed. For example, in various embodiments, the first and second values are determined by filtering a plurality of values recorded in the respective time-intervals. In this case, the answer to the question can be classified by comparing the first value with the second value.

Alternatively, an identifying value of the pupillary diameter can be determined during a measurement interval. In particular, this measurement interval has a shorter duration than the duration of the first and possibly of the second time-interval, for example, less than 0.5 s. For example, the value can be determined by filtering a plurality of values recorded during the measurement interval. Subsequently, whether the pupil diameter has been reduced can be detected by comparing the value with either a fixed or a dynamically determined threshold (e.g., the latter could be determined as a function of the recorded values). In the case in which the pupillary diameter presents a reduction, the instant in which this reduction is present can also be detected. Therefore, in this case, the answer to the question can be classified by detecting whether the instant is in the first time-interval or (if provided) in the second time-interval.

Various embodiments concern a relative system capable of implementing the method. In particular, this system includes:
at least one visual target placed proximally to a subject, and at least one visual target placed distally with respect to said subject,
a recording device for monitoring the pupil diameter, such as a video camera or an eye-tracker,
a processing unit, such as a computer, operatively connected to the recording device and configured for:
   a) recording an identifying value of the pupillary diameter of the subject during a first time-interval and (possibly) a second time-interval,
   b) classifying the answer to the question as either affirmative or negative by processing said value, i.e., detecting if pupillary diameter presents a reduction in the first time-interval or (possibly) in the second time-interval.

### BRIEF DESCRIPTION OF THE DRAWINGS

The invention will now be described, by way of example only, with reference to the attached figures, wherein:

FIG. 1 shows a schematic representation of a system for ocular tracking of a subject according to an embodiment of the present description;

FIG. 2 illustrates a flowchart of a first embodiment of a method for detecting voluntary binary responses of a subject;

FIG. 3 shows an example of the signal detected by the system of FIG. 1 and processed with the method of FIG. 2;

FIG. 4 illustrates a flowchart of a second possible embodiment of a method for detecting voluntary binary responses of a subject;

FIG. 5 shows an example of the signal detected by the system of FIG. 1 and processed with the method of FIG. 4.

### DETAILED DESCRIPTION OF PREFERRED EMBODIMENTS

In the following description, a number of specific details are provided to allow a thorough understanding of embodiments. The embodiments can be put into practice without one or more of the specific details or with other methods, components, materials, etc. In other cases, well-known structures, materials or operations are not shown or described to avoid confusing aspects of the embodiments.

Reference throughout the present disclosure to “one embodiment” or “an embodiment” indicates that a particular appearance, structure or characteristic described with reference to the embodiment is included in at least one embodiment. Thus, the expressions “in one (adjective numeral) embodiment” or “in an (indefinite article) embodiment” at various points throughout the present disclosure do not necessarily all refer to the same embodiment. Furthermore, the particular aspects, structures or characteristics may be combined in any suitable manner in one or more embodiments. The titles provided herein are for convenience only and do not interpret the scope or purpose of the embodiments.

In one or more embodiments, the term “ocular tracking method” refers to a method capable of recording the dilation and contraction of a subject's pupils.

In one or more embodiments, the term “voluntary binary responses” means voluntary affirmative (YES) and negative (NO) replies.

A monitoring system of cognitive activity, based on the ocular tracking of a subject is, for example, described in the document EP 1 164 919 B1. Operationally, the method described in this document, which provides a pupillary analysis during the execution of cognitive tasks, requires complex calibration steps of the measuring instruments on the individual subject.

The method and the system subject of the present description, instead, are based on the recognition of the pupillary constriction which occurs physiologically and automatically during the accommodation process that accompanies focusing of proximal objects with respect to distal objects (a phenomenon also known as pupillary response to accommodation, RPA, direct or consensual).

The method of the present description, without requiring any type of training, without inducing cognitive stress in the subject, or requiring complex individual calibrations of the measurement systems, allows acquisition of binary responses from the subject (of the “YES/NO” type), on the basis of decoding of the pupillary constriction that occurs during the process of ocular accommodation in a given time-interval.

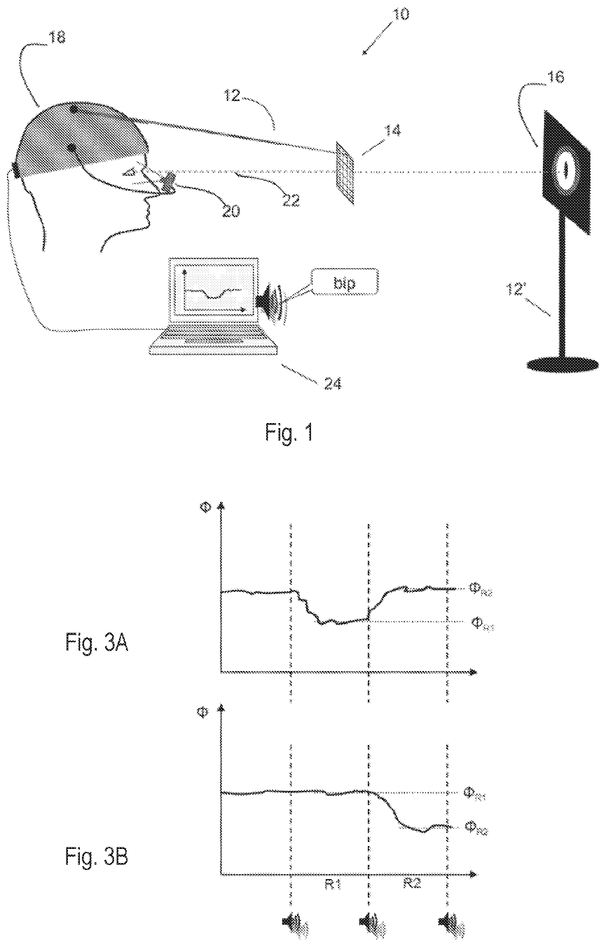

FIG. 1 schematically represents an embodiment of a system 10 for detecting binary responses of a subject, for example, a subject suffering from partial or total paralysis of the skeletal muscles.

In the embodiment in question, the system 10 comprises at least one visual target 14 positioned proximally to the subject and a visual target 16 arranged distally to the subject, i.e. the target 14 is placed between the subject and the target 16. In various embodiments, the proximal visual target 14 and the distal visual target 16 are preferably aligned with each other and with the patient's visual axis 22. For example, in various embodiments, the distal visual target 16 and the proximal visual target 14 can be aligned with the visual axis of the patient's dominant eye.

For example, the distal visual target 16 can be represented by a two-dimensional or three-dimensional object. This visual target can cover a large portion of the subject's field of vision, and is preferably arranged in a position that meets the visual axis 22 of the subject. For example, in various embodiments, the distal target 16 has a particular dimension and is positioned in such a way that it extends from ±15 degrees to ±60 degrees on the horizontal axis and from ±10 degrees to ±40 degrees on the vertical axis with respect to the visual axis 22 of the subject. For example, the distal target 16 may be a wall of the room, or preferably an object having some level of contrast and/or detail, such as a picture, a poster, etc., to facilitate focusing.

The proximal visual target 14 may have a surface that is at least partially transparent. For example, the proximal visual target 14 can be represented by a grid. The transparency, at least partial, of the proximal visual target 14 gives the method the advantage of allowing the subject to visualize the distal target 16 without the need to move the pupil. The semitransparency of the proximal target 14 thus permits visualization of the distal target 16 without the need to shift the visual axis 22, which is advantageous for CLIS patients who are unable to do so. In general, the proximal target 14 has visual structuring/contrast elements to facilitate focusing. For example, the proximal target 14 may be a glass or plexiglass plate to which a design is applied. For example, the drawing can be a grid comprising horizontal and vertical lines, or other regular patterns with lines (for example, with rhombuses or hexagons). Preferably, the size of the spaces in the pattern, for example, the size of the squares/diamonds/hexagons is from 3 to 15 mm. The thickness of the lines can be from 0.3 to 2 mm. In various embodiments, the color of the lines is uniform and the color of the lines is preferably light (e.g. white).

In one or more embodiments, in particular in the event that the subject has a residual possibility of communicating, the proximal target 14 and the distal target 16 can be represented by common objects (with a two- or three-dimensional surface).

In various embodiments, the system 10 may comprise support means 12 and/or 12′ for presenting the target 14 and/or 16. For example, in the embodiment considered, a bar (preferably telescopic) 12 is used that is applied directly to a helmet 18 worn by the subject, wherein said bar 12 supports the target 14. In general, other support means 12 can also be used which allow the height of the proximal target 14 (in particular with respect to the eyes of the subject) to be adjusted, as well as the distance of the proximal target 14 from the subject's eyes, which allows positioning of the proximal target 14 along the visual axis 22 of the subject and facilitating focusing without straining the eyes, which could occur, for example, if the proximal target 14 was too close to the subject's eyes. The distal visual target 16 can be supported by a support post 12′, preferably with adjustable height.

In the embodiment considered, the proximal target 14 is presented at a distance of 20 to 60 cm from the subject's eyes, preferably from 20 to 40 cm. On the other hand, the distal target 16 can be placed at a distance of 2 to 6 m from the subject's eyes, preferably from 5 to 6 m.

In the embodiment considered, the system also comprises a recording device 20. In general, this device 20 is positioned in such a way as to record the pupil diameter and can be placed in front of the subject without obstructing the vision. This device 20 can be represented, for example, by a video camera, such as a webcam, or an “eye tracker”, such as, for example, EyeTribe tracker, EyeTribe, Denmark; or TX300 Tobii, Sweden. In various embodiments, the recording device 20 allows (automatically or manually) focusing on one or both of the subject's eyes and it records the constriction and dilation movements of the pupil.

In the embodiment in question, the recording device 20 is connected to a computer 24 by means of connections with and/or without wires. In general, the recording device 20 and the computer 24 can also be integrated into a single device. For example, the device 20 and the computer 24 can be a laptop, tablet or smartphone.

In the embodiment considered, the computer 24 is then configured to analyze the pupil diameter to classify the subject's response. In general, in the case where an eye-tracker 20 is used, this device can already provide the pupil diameter. On the other hand, in the case where a camera 20 is used, the computer 24 can analyze the images acquired by this camera and obtain the pupillary diameter by processing these images.

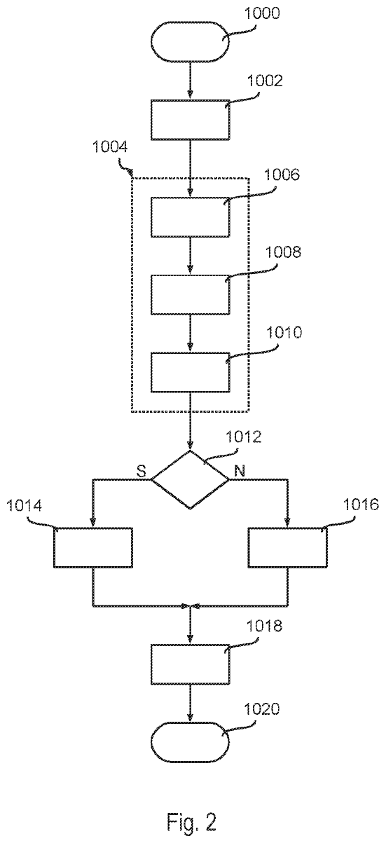

FIG. 2 shows a flowchart of an embodiment of a method for detecting voluntary binary responses of a subject.

Operatively, after a beginning step 1000, the operator poses a question to the subject, at step 1002, formulating a question that requires an affirmative (YES) or negative (NO) response. The method can be based, for example, on the psychophysical design called two-alternative forced choice (2AFC) and its variations.

The task that the subject must perform just requires moving his/her gaze from the distal visual target 16 to the proximal visual target 14 in one of two consecutive time-intervals R1 or R2 depending on the type of response (YES/NO).

For example, the subject can be instructed to shift his/her gaze from the distal visual target 16 to the proximal visual target 14 in the first time-interval R1 if the intention is to communicate an affirmative answer (YES). The subject could be instructed in such a way that if his/her intention is to communicate a negative response (NO), shifting of the gaze from the distal visual target 16 to the proximal visual target 14 occurs in the second time-interval R2.

In general, before the start of the real task, the subject can also be instructed in step 1002 to perform a test, for example, to communicate an affirmative “YES” response, for example, by shifting the attention from the distal visual target to the proximal visual target in the first time-interval R1. In addition to, or alternatively, the subject can be instructed to communicate a negative “NO” response, for example, by shifting the attention from the distal visual target to the proximal visual target in the second time-interval R2.

In various embodiments, the computer 24 is then configured to detect, at step 1004, the fact that the pupillary diameter presents a reduction in the first time-interval R1 or in the second time-interval R2.

For example, in the considered embodiment, monitoring and recording of the pupil diameter is represented in block 1006. For this purpose, a keyboard key could be pressed, to signal to the computer 24 to start the above-mentioned analysis and recording. This event corresponds to the beginning of the time-interval named R1.

In the considered embodiment, the computer 24 detects a value ϕ_{R1} (step 1008), corresponding to a certain pupil diameter, measured during the first interval R1, and another value ϕ_{R2} (step 1010), corresponding to the pupil diameter measured during the second interval R2.

In various embodiments, the computer 24 may communicate the starting and ending points of both intervals R1 and R2, for example, by visual and/or acoustic stimuli (see FIGS. 3A and 3B). In general, as shown in FIGS. 3A and 3B, the starting point of the second time-interval R2 may also correspond to the end of the first time-interval R1. The start and finish for both time-intervals may also be indicated by the operator.

In various embodiments, the time-intervals R1 and R2 may have a constant temporal duration. For example, the duration of each time-interval could be from 2 to 10 s, resulting in an overall duration of 4-20 s, for example 4-10 sec. In one or more embodiments, the first and second time-intervals R1 and R1 have the same duration.

Therefore, during the recording in steps 1008 and 1010, the subject “interfaces” with others communicating his/her intention (YES/NO), by simply shifting his/her gaze. FIGS. 3A and 3B show, respectively, the pupillary diameter trend ϕ for a “YES” response and a “NO” response. Here the subject communicates his intention via binary answers (YES/NO) by:
focusing on the proximal target 14 in the first time-interval R1 and on the distal target 16 in the second time-interval R2 (FIG. 3A, positive answer);
focusing on the distal target 16 in the first time-interval R1 and on the proximal target in the second time-interval R2 (FIG. 3B, negative answer).

In the embodiment considered, the computer 24, in 1012, is configured to check if the pupillary diameter is reduced in the first time-interval R1 or in the second interval R2. For example, since the diameter reduction only appears in one time-interval, the computer 24 can check if the pupillary diameter ϕ_{R1} is less than the pupillary diameter ϕ_{R2}.

For example, whenever there is a diameter reduction in the first interval R1, i.e. the pupillary diameter ϕ_{R1} is less than the pupillary diameter ϕ_{R2} (output “S” in step 1012), the computer 24 may classify the answer (step 1014) with a first state (for example, a “YES” response).

On the other hand, whenever the diameter is found to be reduced in the second interval R2, i.e. the pupillary diameter ϕ_{R1} is more than the pupillary diameter ϕ_{R2} (output “N” in step 1012), the computer 24 may classify the answer (step 1016) with a second state, for example a “NO” response.

In various possible embodiments, the computer 24 may indicate the classification result in step 1018 and the method ends at 1020. For example, the computer 24 may show, in step 1018, the results on the screen of the computer 24 and/or produce a particular sound.

As previously described, in the embodiment considered, the computer 24 determines the values ϕ_{R1} e ϕ_{R2}, respectively associated with the pupillary diameters in the time intervals R1 and R2.

With this aim, the computer 24 may, for example, process the video recording from the connected webcam 20, in order to determine the instant pupillary diameter ϕ.

In the simplest case, the computer 24 may use the value ϕ obtained at the end of the interval R1 for the pupillary diameter ϕ_{R1}, and the value ϕ obtained at the end of the interval R2 for the pupillary diameter ϕ_{R2}. In general, the computer 24 may choose any representative value for the diameter, for example, a value selected from the middle of the corresponding time-interval.

In various embodiments, the computer 24 may also determine the values ϕ_{R1} and ϕ_{R2} by considering a plurality of values from the respective time interval. For example, in various embodiments, the values ϕ_{R1} and ϕ_{R2} are determined, respectively, by means of filtering the values ϕ in the intervals R1 and R2. For example, in various embodiments, the average value for all recorded diameters in the corresponding time-interval R1 and R2 can be used, or the moving average. In general, this filtering may also consider just a limited number of recorded values ϕ, for example, the final twenty recorded values.

In various embodiments, the filtering method may also involve artefact-removal steps.

Therefore, in the embodiment considered, the computer 24 processes and analyses the recorded pupillary diameters (I), in order to detect the interval R1/R2 in which the subject focused on the proximal target 14. In other words, the computer 24 determines the sequence of the focused targets and associates the subject's presumed answer (binary: YES/NO).

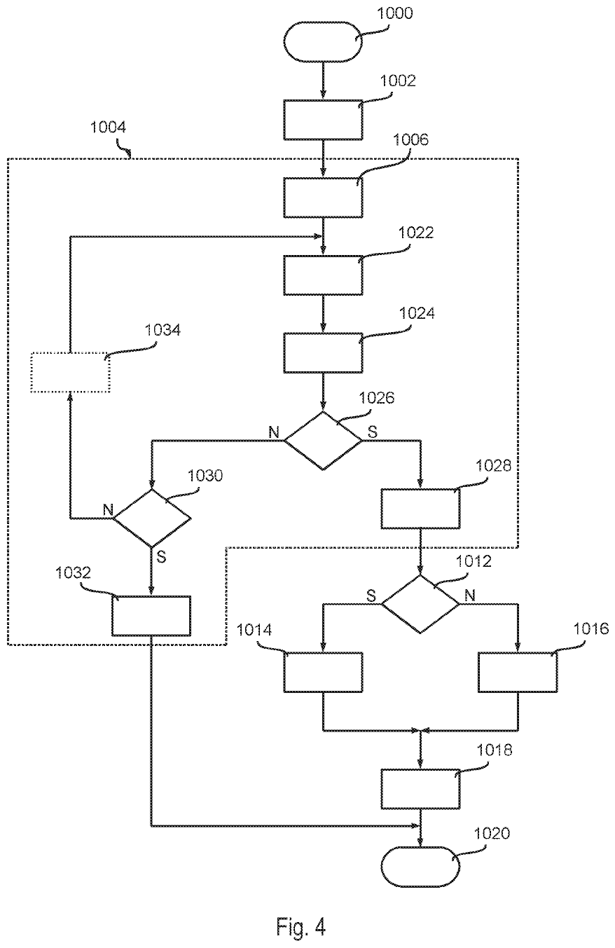

FIG. 4 shows a second embodiment of the invention.

Basically, as mentioned above with reference to FIG. 2, the subject stares at the distal target 16 when a task is given (step 1002) and shifts his/her gaze onto the proximal target during the first time-interval R1 or during the second time-interval R2.

Therefore, also in the considered embodiment, the computer 24 records the pupillary diameter ϕ during the intervals R1 and R2, and processes the recorded values, in order to detect the interval R1/R2 in which a reduced pupillary diameter ϕ is presented.

However, instead of determining the representative values ϕ_{R1} and ϕ_{R2} for the pupillary diameter in each interval, the computer 24 detects—in step 1004—the approximate instant corresponding to the pupillary diameter reduction, i.e. the instant when the subject focuses on the proximal target 14.

Therefore, in the considered embodiment, the computer 24 may verify—in step 1012—if this instant is in the first interval R1 or in the second interval R2. For example, if the reduction occurs in the first interval R1 (output “S” in step 1012), the computer 24 carries out a checking step, (step 1014), classifying the output as “YES”. On the other hand, if the reduction occurs in the second interval R2 (output “N” in step 1012), the computer 24 proceeds again to step 1016, in which the response is classified with a second state, for example, a “NO” response.

In particular, in order to detect the instant corresponding to the pupillary diameter reduction, recording of the pupillary diameter is activated (step 1006).

Subsequently, the computer 24 records—in step 1022—a value ϕ_{R}, corresponding to the pupil diameter. This value ϕ_{R} may, in general, be equal to the instant value of the recorded diameter value ϕ, or may be obtained as function of a plurality of recorded values ϕ. In various embodiments, an acquisition window is used, involving several recorded values for a measurement interval. In various embodiments, this measurement interval is less than the duration of the intervals R1 and R2, or rather at least 10% less than the duration of the intervals R1 and R2. For example, in various embodiments, the duration of the measurement interval is from 0.1 to 0.5 s, for example from 0.2 to 0.3 s.

In general, the computer 24 may also further process the values ϕ_{R}. For example, by analyzing a plurality of ϕ_{R} values, artefacts originating from eyelid movements may be collected and removed by the computer 24. The deleted values ϕ_{R} may be replaced by interpolated (linear) values from adjacent values.

In the embodiment considered, the computer 24 determines a threshold value S in step 1024. This threshold may be fixed or dynamically determined as a function of the recorded values ϕ.

For example, in various embodiments, this threshold is determined during a calibration phase, when the subject is asked to stare at the distal target, for example when the question is posed. For example, the threshold S may correspond to a certain percentage (e.g. from 90-95%) of the pupillary diameter value ϕ, when the subject focuses on the distal target 16. Alternatively, the threshold S may also be calculated by subtracting a fixed value from the pupillary diameter ϕ when the subject focuses on the distal target 16.

Instead, the computer 24 may also calculate the threshold S directly as a function of the acquired values ϕ_{R}. For example, in various embodiments, a filter is used that calculates the mean or the moving mean ϕ_{M} on several values ϕ_{R}. Subsequently, the computer 24 uses a certain percentage of this value ϕ_{M} (for example 90-95%) as the threshold S. Alternately, the threshold S can be calculated by subtracting a fixed value from the value ϕ_{M}.

In the considered embodiment, at step 1026, the computer 24 compares the value ϕ_{R} with the threshold S.

In the event that the value ϕ_{R} is less than the threshold S (output “S” of the step 1026), the computer 24 stores—at step 1028—the time passed from the start of the recording at step 1006. Subsequently, there is a step 1012 to check if this time indicates that the pupillary diameter reduction occurred in the first interval R1 or the second interval R2; for example, by comparing the computed time with the duration of the interval R1. The method then proceeds as described in FIG. 2.

On the other hand, if the value ϕ_{R} is bigger than the threshold S (output “N” in step 1026), the computer 24 proceeds to step 1030 to check if a maximum time has passed. In the considered embodiment, this maximum time corresponds to the sum of the durations of the intervals R1 and R2.

In the event that the maximum time has not passed (output “N” in step 1030), the method returns to step 1022 to perform another monitoring cycle.

On the other hand, if the maximum time has passed (output “S” of the step 1030), the computer signals an error at a step 1032, since no decrease in the pupil diameter has been detected. In this case, the method ends at step 1020.

In various embodiments, rather than simply verifying the instant value of ϕ_{R} at step 1026, the computer 24 may also verify whether ϕ_{R} is lower than the threshold S, within a given verification window lasting, for e.g., 0.5 s.

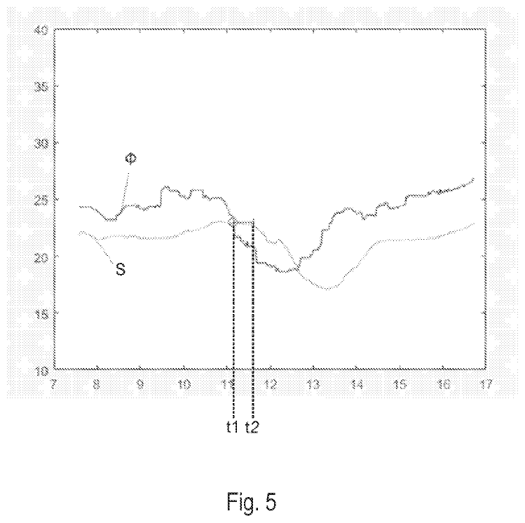

For example, FIG. 5 shows a possible time course of the pupil diameter values ϕ and of the threshold S (which is computed based on filtered ϕ_{R} values), during a certain measuring-interval. In particular, in this example, the value ϕ decreases below the threshold S at time t1=11.2 s. Thus, by using a verification window of 0.5 s, the computer 24 detects that, at time t2=11.7 s, the values ϕ_{R} were below the threshold S for the final 0.5 s, thus proceeding to step 1028. For example, assuming that both time-intervals R1 and R2 have a 10-s duration, the decrease in pupillary size occurs during the interval R2.

Therefore, from a practical point of view, the acquired ϕ_{R} values can be consecutively stored in a buffer. For example, the time distance between two stored ϕ_{R} values may be equal to 0.2 s. For example, for this purpose, a waiting step 1034 may be inserted between the verification step 1030 and the acquisition step 1022. For example, step 1034 may be used to determine the value ϕ_{R} by filtering ϕ values over a corresponding time-period. Step 1034 may also be used to indicate the end of the interval R1 and/or the beginning of the interval R2.

In general, the buffer size can be dimensioned to include at least the values of a verification window. The buffer may be even larger, for example, to allow for computation of the threshold S based on the values ϕ_{R}. For example, in various embodiments the buffer may store the ten last ϕ_{R} values. For example, the seven oldest values may be used to compute the threshold S (e.g., by using a weighted (moving) average), and the three most recent values may be compared to this threshold S, at step 1026.

In general, at step 1026, the computer 24 could also check whether the last stored ϕ_{R} values present a negative gradient, which would indicate that the curve has a negative slope.

The method and the system described here present many advantages compared to known methods for detecting voluntary binary responses from locked-in patients.

One advantage lies in the ease and robustness of the detection: the changes in pupil diameter during the accommodation process are very large, in the order of few millimetres (mm).

In addition, these changes are stereotyped and, therefore, can be easily detected and classified, with no need for complex and calibration of the employed devices on the individual subject.

Another advantage of the present method is that it can interpret the intention of a subject by using a decidedly simplified instrumentation compared to other existing systems for Brain-Computer-Interface, BCI. In fact, the method and the relative system described here do not require expensive equipment or complex implementation.

Moreover, the task that the subject is required to do is very simple and natural and does not require any specific training. This also presents positive implications in terms of appreciation and the collaborative attitude in the tested subjects.

In general, the second interval R2 is purely optional. In fact, in order to classify the answer to a given question, it is sufficient to analyze the pupil diameter ϕ during the interval in which the question is posed and for the first subsequent time-interval. For example, in the embodiments previously considered, the subject gazes at the distal target 16 at the time when the question is posed. Therefore, this time-interval can be used to measure the reference pupil diameter related to the distal target 16. Then (during the subsequent time-interval R1), the subject keeps looking at the distal target (e.g., in the case in which the intended answer is “NO”) or looks at the proximal target (e.g., in case in which the intended answer is “YES”. Therefore, the second interval R2 constitutes a verification interval to detect a response that should be opposite to that detected in interval R1.

For example, in the embodiment described with reference to FIG. 3, the lack of interval R2 may be accounted for by omitting step 1010 and by computing the value ϕ_{R2} as a function of the ϕ values acquired while posing the question, i.e., in this case, the value ϕ_{R2} is representative of the pupil diameter during the time-interval in which the question is posed.

Even in this case, the computer 24 may check—at step 1012—whether the pupil diameter presents a reduction within the interval R1. For example, to this aim, the computer 24 may compare the pupil diameter ϕ_{R1} with the pupil diameter ϕ_{R2}. For example, to this aim, the computer 24 may compute a threshold value ϕ′_{R2} as a function of the pupil diameter ϕ_{R2}, e.g., by scaling the value ϕ_{R2} or by subtracting a predefined value. Thus, if a reduction in pupil diameter takes place in the interval R1, i.e., the pupil diameter ϕ_{R1} drops below the threshold ϕ′_{R2} (output “S” at verification step 1012), at step 1014 the computer 24 may classify the answer as the first state, which, for example, identifies the answer “YES”. Conversely, if a reduction does not take place in the time-interval R1, i.e., the pupil diameter ϕ_{R1} remains above the threshold ϕ′_{R2} (output “N” at verification step 1012), at step 1016 the computer 24 may classify the answer as the second state, which, for example, identifies the answer “NO”.

On the other hand, the embodiment described with reference to FIG. 4 may already detect a decrease in pupil diameter at step 1026. Therefore, if a reduction at step 1026 is not observed and step 1030 detects that the maximum time has expired, the method may jump directly to step 1016, rather than proceeding with step 1032, in order to classify the answer as the second state, which, for example, identifies the answer “NO”. Instead, if a reduction is detected at step 1026, the method may omit step 1028 and step 1012 and proceed directly to step 1014, in order to classify the answer as the first state, which, for example, identifies the answer “YES”.

Therefore, the present description provides a method and a system for detecting binary responses of a subject, which has the advantage of being simple, of not requiring complex calibration of measurements and methods, and of not requiring expensive equipment. Moreover, the method has the advantage that no cognitive stress is imposed on the subject, which may occur when complex tasks have to be learned.

### REFERENCES

Stoll et al, Pupil responses allow communication in locked-in syndrome patients; Curr Biol 23:R647-648, 2013.
Binda et al, Pupil size reflects the focus of feature-based attention, J Neurophysiol 112:3046-3052, 2014.
Mathôt et al, The Mind-Writing Pupil: A Human-Computer Interface Based on Decoding of Covert Attention through Pupillometry. Plos One 2016; DOI:10.1371/journal.pone.0148805

1. Method of detecting voluntary binary responses of a subject affected by partial or total paralysis of skeletal muscles, comprising:
arranging at least one visual target at a proximal position with respect to the subject and at least one visual target at a distal position with respect to the subject,
asking the subject at least one question requiring a positive or a negative response,
recording a value indicative of the pupil diameter of said subject during a first time-interval, wherein said pupil diameter is susceptible to reduction as the attention is shifted, due to focusing, from said distal visual target to said proximal visual target, and
classifying the response to said question as positive or negative by means of processing said recorded value by detecting the fact that said pupil diameter presents a reduction in said first time-interval.

2. Method according to claim 1, comprising:
recording a value indicative of the pupil diameter of said subject during a second time-interval, and
classifying the response to said question as positive or negative by means of processing of said recorded value by detecting the fact that said pupil diameter presents a reduction in said first time-interval or in said second time-interval.

3. Method according to claim 1, wherein said visual target arranged at a proximal position with respect to the subject is arranged at a distance of 20 to 60 cm from the eyes of the subject, preferably from 20 to 40 cm.

4. Method according to claim 1, wherein said visual target arranged at a distal position with respect to the subject is arranged at a distance of 2 to 6 m from the eyes of the subject, preferably from 5 to 6 m.

5. Method according to claim 1, wherein said visual target arranged at a proximal position, and said visual target arranged at a distal position, are aligned with the visual axis of the subject.

6. Method according to claim 1, wherein said visual target arranged at a proximal position is an object that is at least partially transparent, with a regular pattern, such as a grid.

7. Method according to claim 1, comprising:
determining a first value indicative of the pupil diameter during said first time-interval as a function of said recorded value, and determining a second value indicative of the pupil diameter during the time-interval when the subject is asked the question, or said second time-interval as a function of said recorded value, and
classifying the response to said question as positive or negative by comparing said first value with said second value.

8. Method according to claim 1, comprising:
determining a third value indicative of the pupil diameter during a measurement interval as a function of said recorded value, wherein said measurement interval is less than said first time-interval,
determining a threshold,
detecting the instant when said pupil diameter presents a reduction by comparing said third value with said threshold, and
classifying the response to said question as positive or negative by detecting whether said instant is in said first time-interval.

9. Method according to claim 7, wherein said first, second and/or third value is determined by filtering a plurality of said recorded values.

10. Method according to claim 1, wherein said first time-interval, and possibly said second time-interval, each have a duration of 2 to 10 seconds, preferably from 2 to 5 seconds.

11. System for detecting voluntary binary responses of a subject affected by partial or total paralysis of skeletal muscles, comprising:
at least one visual target arranged at a proximal position with respect to the subject, and at least one visual target arranged at a distal position with respect to the subject,
a recording device for monitoring the pupil diameter, such as for example a video-camera or an eye-tracker, and
a processing device, such as for example a computer, operatively connected to the recording device and configured for:
a) recording a value indicative of the pupil diameter of said subject during a first time-interval, and
b) classifying the response to said question as positive or negative by means of processing said recorded value, by detecting the fact that said pupil diameter presents a reduction in said first time-interval.

12. System according to claim 11, wherein said visual target arranged at a proximal position with respect to the subject is arranged at a distance of 20 to 60 cm from the eyes of the subject, preferably from 20 to 40 cm.

13. System according to claim 11, wherein said visual target arranged at a distal position with respect to the subject is arranged at a distance of 2 to 6 m from the eyes of the subject, preferably from 5 to 6 m.

14. System according to claim 11, wherein said visual target arranged at a proximal position and said visual target arranged at a distal position are aligned with the visual axis of the subject.

15. System according to claim 11, wherein said visual target arranged at a proximal position is an object that is at least partially transparent, with a regular pattern, such as a grid.
